(19) United States
(12) Patent Application Publication
(10) Pub. No.: US 2004/0158311 A1
(43) Pub. Date: Aug. 12, 2004

(54) Intravascular stent

(76) Inventors: Steven W. Berhow, St. Michael, MN (US); Zhenghui Cheng, Shenzhen (CN); Gordon Hocking, Tokyo (JP)

Correspondence Address: BRIGGS AND MORGAN, P.A., 2400 IDS CENTER, MINNEAPOLIS, MN, 55402, US

(21) Appl. No.: 10/360,362

(22) Filed: Feb. 6, 2003

(51) Int. Cl. A61F 2/06

(52) U.S. Cl. 623/1.15

(57) ABSTRACT

A stent having a stent body and a embolic body is disclosed. The stent body is collapsible for insertion into a vessel or other lumen within a patient and is radially expanded at the site of an aneurysm so that the embolic body is received within the aneurysm to support and/or promote clotting within the aneurysm. The embolic body may also be collapsible and radially expandable.

### BACKGROUND OF THE INVENTION

1. Field of the Invention

The present invention relates to an intra-vascular apparatus and method for the treatment of aneurysms and, particularly, an intra-vascular apparatus and method for the treatment of saccular aneurysms.

2. Brief Description of the Related Art

The rupturing of aneurysms is a serious medical problem. The mortality rate for patients having a ruptured aneurysm is about 10% within the first day of rupture and about 25% within three months. Even for those who survive the initial hemorrhage, there is frequent neurological damage which impairs the patient's quality of life.

Aneurysms may occur at a variety of locations within the arteries of the human body. Generally, aneurysms are sack-like bulges in the arterial wall which extend outward from an arterial wall thinning the wall of the artery. Aneurysms may occur at any location along an artery but frequently occur at bifurcations in the artery or at locations which have been weakened by trauma, illness, or otherwise. With sacculated aneurysms, the aneurysm typically forms a neck at the juncture with the artery and is capped by a dome.

Blood circulates under pressure through the aneurysm and can cause the aneurysms to enlarge and weaken. The morphology of the tissue of an aneurysm is distinct from the associated vessel, the arterial internal elastic lamina disappears at the base of the neck, the sack wall, and connective tissue replaces smooth-muscle cells. These changes generally weaken the aneurysm and make the aneurysm less elastic and susceptible to the pressure changes created by the beating of the heart. Accordingly, there is a significant risk of an aneurysm rupturing. Frequently, the aneurysm will rupture at the dome which is the region of the aneurysm opposite from the neck. Naturally, therapeutic treatment of aneurysms emphasizes preventing the initial rupture.

A wide variety of techniques for treating aneurysms have evolved. These techniques have frequently involved positioning the tip of a catheter partially into the neck of the aneurysm and depositing various materials within the aneurysm to promote thrombosis. Various configurations of wires are common uses for this purpose. Depending on the particular method being used, a single wire or a plurality of wires may be used to fill the aneurysm. With other techniques, a matrix of biocompatible material is used to promote thrombosis.

When a single wire is used, the wire typically has a length between 0.4 and 2.0 inches and a diameter of between 0.001 and 0.005 inches. The flexible wire loops and tangles randomly as it is packed into the aneurysm. When multiple wires are used, the wires are typically shorter and are frequently configured to become entangled with one another within the aneurysm to prevent the accidental release of one or more wires into the patient's bloodstream. The blood which normally circulates under pressure through the aneurysm will begin to form clots on the tangled wires and, eventually, the clot will enlarge to form an occlusion which seals off the aneurysm from the blood flow. The formation of an occlusion has generally been found to prevent further enlargement and rupture of the occluded aneurysm. However, the insertion of the one or more wires into an aneurysm creates the risk that one or more wires can become dislodged and enter the patient's bloodstream. These dislodged wires can lead to unwanted clot formation in the lumen of a healthy artery that can endanger the well being of the patient. Therefore, a need exists for an apparatus and method which eliminates or reduces the possibility of a dislodged wire. In addition, even if a wire is not fully dislodged from the aneurysm, a wire may partially dislodge from the aneurysm to extend into the artery. Blood flowing through the artery past the wire can form a clot on the protruding end of the wire. This clot could separate from the wire end and, potentially, cause a stroke or embolism. Statistical results predict that as many as 5% of the patients treated by this technique will suffer complications caused by the wire extending through the aneurysm neck and into the artery. Therefore, a need exists for an apparatus and method for the treatment of embolisms that would reduce the potential for embolisms or stroke as a result of the procedure.

Further, regions surrounding aneurysms can be susceptible to stenosis. The stenosis may be treated by procedures such as balloon angioplasty to break loose the material clogging the artery to permit the efficient flow of blood past the aneurysm. However, treated stenotic sites frequently narrow or close again within six months after balloon angioplasty, through a phenomenon called restenosis.

In an effort to prevent restenosis or treat an aneurysm without requiring surgery, short flexible cylinders or scaffolds made of metal or polymers are often placed into a vessel to maintain or improve blood flow. Referred to as stents, various types of these devices are widely used for reinforcing diseased blood vessels, for opening occluded blood vessels, and for defining an internal lumen bulkhead to relieve pressure in an aneurysm. The stents allow blood to flow through the vessels at an improved rate while providing the desired lumen opening or structural integrity lost by the damaged vessels. Some stents are expanded to the proper size by inflating a balloon catheter, referred to as “balloon expandable” stents, while others are designed to elastically resist compression in a “self-expanding” manner.

Balloon expandable stents and self-expanding stents are generally delivered in a cylindrical form, crimped to a smaller diameter around some type of catheter-based delivery system. When positioned at a desired site within the lesion, they are expanded by a balloon or allowed to “self-expand” to the desired diameter. However, many vessels are too small to accept a stent shaped in a cylinder during delivery.

Another type of stent is formed of a wire that has a relaxed cylindrical shape, yet can be stretched into a linear shape for delivery through a much smaller catheter than any stent delivered in cylindrical form. The basic design of such a “linear” stent is described in U.S. Pat. No. 4,512,338, issued Apr. 23, 1985 to Balko.

Balko discloses a shape memory nitinol wire, shaped in its parent phase into a coil of adjacent wire loops, then cooled to its martensite phase and reshaped to a straight shape. The wire is inserted into the vessel with thermal insulation, such that the wire reforms to its coil shape upon the removal of the insulation means, so as to reform the damaged vessel lumen.

However, this basic linear stent does not facilitate thrombus formation to occlude an aneurysm. Moreover, aneurysms may form at a bifurcation where one vessel branches off from another. The basic linear stent is generally ineffective treatment for these bifurcation aneurysms. Therefore, a need exists for an apparatus and method that can be easily delivered to a vascular site through a catheter, that is capable of being atraumatically positioned, providing an element to promote thrombus formation within an aneurysm, and that exhibits sufficient structural integrity and resilience under inward forces.

The apparatus and method of the present invention can meet the above needs and provides additional improvements, advantages and features that will be apparent to those skilled in the art upon review of the following description and the appended drawings.

### SUMMARY OF THE INVENTION

The present invention provides a stent and stenting system having a stent body to hold a vessel passageway open and an embolic body to support an aneurysm by extending into and either or both of, supporting the aneurysm and promoting the occlusion of an aneurysm. An embodiment of a stent or stenting system in accordance with the present invention can be delivered in a linear fashion through a catheter and can be reconfigured in an expanded configuration upon deployment from the catheter. An embodiment of a stent in accordance with the present invention can treat a saccular aneurysm, by providing a stent which includes an embolic body to extend into the aneurysm. In addition, an embodiment of a stent in accordance with the present invention can treat a bifurcation aneurysm, by providing a stent which includes extensions that are received by the branches of a bifurcation and providing embolic body which extends into the aneurysm.

A stent to stabilize an aneurysm in accordance with the present invention can include a stent body and an embolic body. The stent body having an outer surface and shaped to be received within a lumen of a patient. In one aspect, the stent body has a cylindrical shape. In another aspect, the stent body can have a cylindrical shape with a bifurcation at one end. The stent body may be configured in at least a collapsed configuration for insertion into the lumen of the patient and an expanded configuration. The embolic body may be secured to the stent body. The embolic body generally extends from the outer surface of the stent body to be received within an aneurysm when the stent is configured in an expanded configuration. In one aspect, the stent of the present invention may include an embolic body defining a cavity within the embolic body. When the stent includes a cavity, a coagulation promoting material can be secured within the cavity.

### BRIEF DESCRIPTION OF THE DRAWINGS

FIG. 1 illustrates a perspective view of an embodiment of a stent in accordance with the present invention;

FIG. 2 illustrates a perspective view of another embodiment of a stent in accordance with the present invention;

FIG. 3 illustrates a perspective view of a stenting system in accordance with the present invention.

FIG. 4 illustrates a side view of an embodiment of a stent in accordance with the present invention;

FIG. 5 illustrates a top view of an embodiment of a stent in accordance with the present invention;

FIG. 6 illustrates a side view of a cross-section through line 5-5 of the stent illustrated in FIG. 5;

FIG. 7 illustrates an embodiment of a stent in accordance with the present invention in a compressed configuration within a catheter; and

FIG. 8 illustrates an embodiment of a stent in accordance with the present invention with the embolic body secured within a saccular aneurysm.

All figures are drawn for ease of explanation of the basic teachings of the present invention only; the extensions of the figures with respect to number, position, relationship and dimensions of the parts to form the preferred embodiment will be explained or will be evident to those skilled in the art after the following description has been read and understood. Further, the exact dimensions and dimensional proportions to conform to specific force, weight, strength, and similar requirements will likewise be evident to those skilled in the art after the following description has been read and understood.

Where used in various figures or on multiple occasions within the same figures, the same numerals generally designate the same or similar parts. Furthermore, when the terms “vertical,” “horizontal,” “top,” “bottom,” “right,” “left,” “forward,” “rear,” “first,” “second,” “inside,” “outside,” and similar terms are used, the terms should be understood to reference only the structure shown in the drawings as it would generally appear to a person viewing the drawings and utilized only to facilitate describing the illustrated embodiment.

### DETAILED DESCRIPTION OF THE INVENTION

The following discussion relates predominantly to embodiments of a stent and a stenting system having an embolic body for stabilizing aneurysms and preventing restenosis for ease of description and clarity. Those skilled in the art will appreciate that the present invention may be used for other indications.

A stent 10 in accordance with the present invention typically includes a stent body 12 having an embolic body 14 extending from the stent body 12. Stent 10 may be more precisely called a stenting system 10 when embolic body 14 and stent body 12 are separable from one another as particularly illustrated in the exemplary embodiment in FIG. 3.

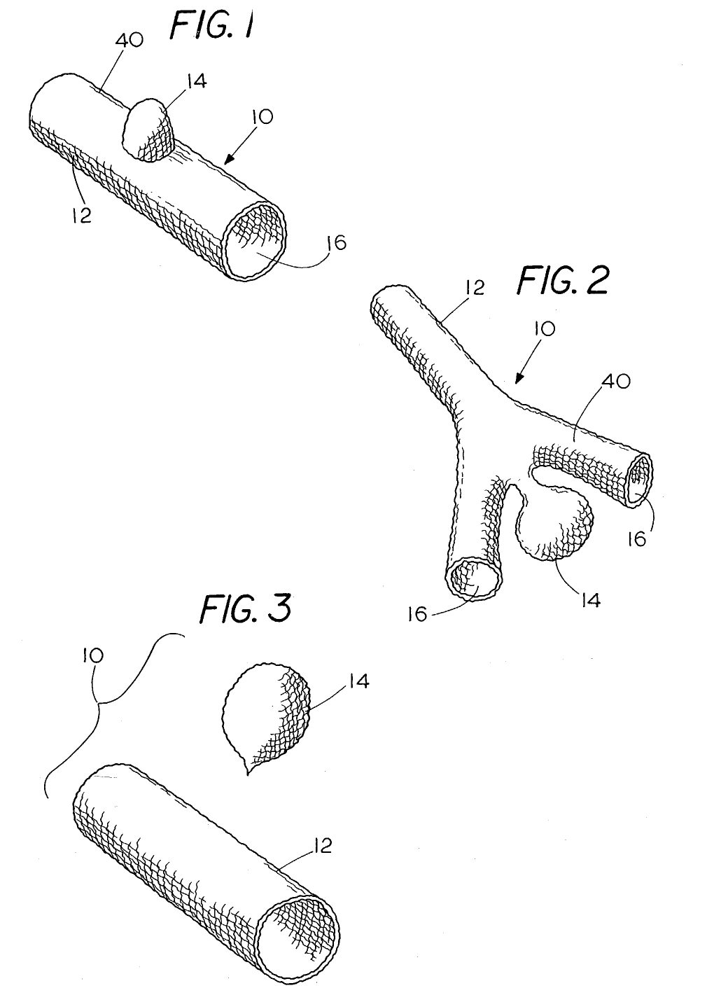

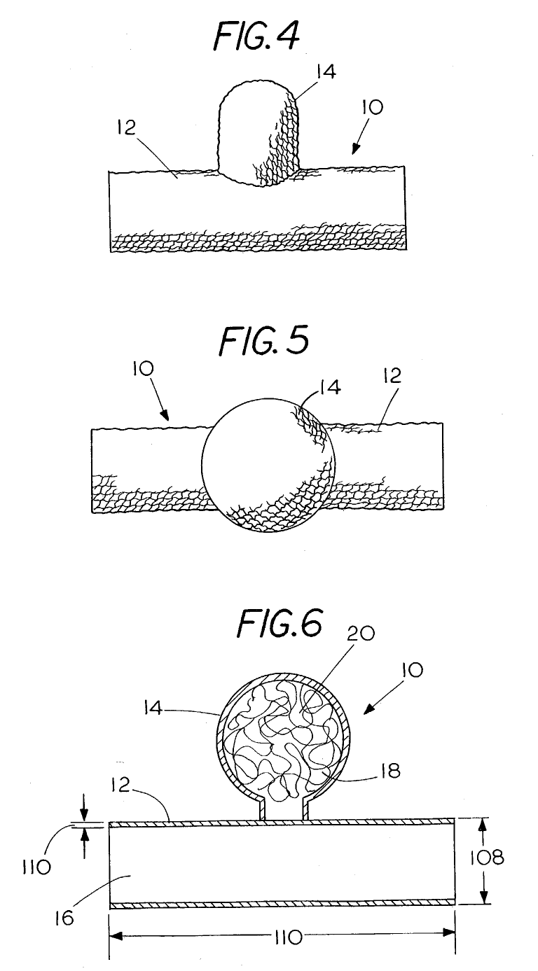

Stent body 12 is typically in the form of an expandable tube defining a lumen 16 through which fluid may flow after implantation of stent 10 within a patient. In one aspect, stent body 12 may bifurcate at one end to support a branched section of vessel 100 around an aneurysm 120 as is generally illustrated in FIG. 2. Stent body 12 can be formed from a mesh, interlocking loop structure or using other structures, generally illustrated as a woven material 40, which allow stent 10 to collapse and expand for insertion into a lumen 102. Stent 10 can be made of a metal, an alloy, a polymer or other material or combination of materials which permit the transition between the collapsed and expanded configurations and which can withstand the forces and stresses which could cause stent 10 to collapse once implanted in the patient. In one aspect, the material may be sufficiently resilient to increase the diameter of the lumen. In another aspect, the material may be sufficiently resilient to maintain the diameter of the lumen. In yet another aspect, the material may be at least in part a radiopaque material to allow at least part of stent 10 to be viewed using fluoroscopy or X-ray techniques. If desired, stent body 12 and embolic body 14 may be made from different materials.

The figures generally illustrate various embodiments of an embolic body 14 suitable for treating saccular aneurysms. Having embolic body 14 secured to stent body 12 can prevent the release and/or protrusion of wires end from an aneurysm 120 into an artery in the treatment of the aneurysm 120. Blood may then still clot on the wire or other material 20 to form an occlusion sealing and/or otherwise stabilizing aneurysm 120.

In one aspect, embolic body 14 is generally illustrated in the figures as a sack secured at the one end to and extending from stent body 12 for exemplary purposes. In another aspect illustrated in FIG. 3, embolic body 14 is not secured to stent body 12 and embolic body 14 is secured within an aneurysm by positioning stent body 12 adjacent to the aneurysm within the vessel. Embolic body 14 may take a number of forms and is typically secured to or integral with stent body 12. When embolic body 14 is configured as an enclosed sack to define a cavity 18, embolic body 14 can contain wires or other material 20 to promote the formation of an embolism within the cavity 18. The cavity 18 may be separated from the lumen of stent body 12 by the woven material of the stent body 12 or embolic element 14, as illustrated in FIG. 6, or the cavity 18 may be open to the lumen of stent body 12, as illustrated in FIG. 8. In other embodiments, embolic body 14 may be any of a variety of elements secured to and extending from stent body 12 which may be positioned within an aneurysm to promote the formation of an embolism. Embolic body 14 preferably comprises a collapsible extension which defines a cavity 18. Embolic body 14 can be formed from a mesh, interlocking loop structure or using other structures which allow stent 10 to collapse and expand for insertion into a lumen and aneurysm. In an expanded configuration, embolic body 14 may substantially fill the volume within aneurysm 120. In one exemplary embodiment, the configuration of embolic body 14 may substantially conform to the inner wall of aneurysm 120. As illustrated in FIG. 8, embolic body 14 is sized to be positioned through a reduced diameter neck portion of aneurysm and to have a greater size in the region of embolic element 14 extending further into aneurysm 120. In one aspect, embolic body 14 may be shaped to conform to the interior shape of the aneurysm 120. In other embodiments, embolic element 14 may be substantially spherical in shape. In still other embodiments, embolic element 14 may be otherwise shaped, such as a cone, a cylinder or an irregular shape, to support the aneurysm and/or to promote the formation of an embolism so as to reduce, as will be recognized by those skilled in the art upon review of the present disclosure. In yet another embodiment, embolic body may be configured as one or more wires secured to stent body 12 to be received within an aneurysm and to promote the formation of an embolism. Other embodiments that will support the aneurysm and/or will promote the formation of an embolism will be evident to those skilled in the art upon review of the present disclosure. Regardless of the shape, embolic body 14 is typically configured to, at least in part, be received within an aneurysm to promote the formation of an embolism and/or to support the inner wall within the aneurysm to stabilize the aneurysm and to reduce the likelihood for rupture.

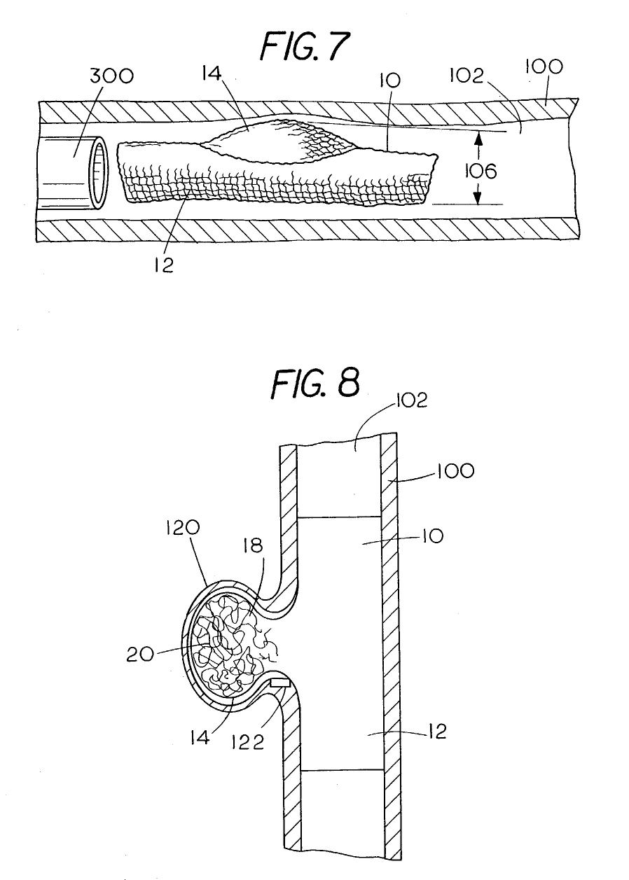

Further, in a collapsed configuration, shown in FIG. 7, embolic body 14 preferably collapses to a size, which in conjunction with the associated collapsed stent body 12, may fit within the lumen of a catheter 300 to permit insertion of stent 10 through the lumen of a blood vessel.

In one embodiment, as generally discussed above, a cavity 18 defined by embolic body 14 contains a coagulation promoting material 20. Coagulation promoting material 20 may one or more wires or may be a polymeric matrix or other material that will promote coagulation of the blood which enters the cavity to form an embolism within the aneurysm. The coagulation promoting material 20 is preferably collapsible to permit the collapse of stent 10 to a size permitting insertion. The coagulation promoting material 20 may to some extent function to support embolic body 14 by expanding in the relaxed configuration to bias against the inner wall of the cavity defined by embolic body 14. The cavity 18 defined by embolic body 14 contains the coagulation promoting material 20 within an aneurysm and thus, can prevent the coagulation promoting material 20 from protruding outwardly from the aneurysm and into the artery and will not provide a location for the undesired formation of clots that may break away and cause an unwanted embolism outside of the aneurysm.

In general, stent 10 is collapsible for insertion into a lumen 102 of a vessel 100, shown in FIGS. 7 and 8. In a collapsed configuration, shown in FIG. 7, stent 10 has an outside diameter 106 that is generally smaller than the diameter of lumen 102. For example, a collapsed stent 10 may have an outside diameter 106 of between about 1.5 millimeters-1.8 millimeters. Once introduced into lumen 102, stent 10 is expanded to a diameter that provides the desire support to or expansion of lumen 102. In one aspect, the expanded stent body diameter 108 may substantially corresponds to the diameter of lumen 102 to support the walls of lumen 102. In another aspect, the expanded diameter may be greater than the diameter of lumen 102 to expand and/or support the walls of lumen 102. Stent 10 may be configured to otherwise function within lumen 204 as will be understood by those skilled in the art upon review of the present disclosure. Typically, stent 10 in the expanded configuration supports the lumen 102 to prevent the recurrence of a stenosis and/or supports an aneurysm 120 to reduce the possibility of the rupturing of aneurysm 120. For example, a collapsed stent 10 may have an outside diameter 106 of between about 1.5 mm-1.8 mm and may have an expanded stent body diameter 108 of between 3.0 mm-4.0 mm. Stent body 12 typically has an overall length 110 of between a few millimeters and a few tenths of a millimeter. The stent body 12 may have a wall thickness 110 of, for example, around a few hundredths of a millimeter. However, the particular dimensions of stent body 12 will be determined by the particular application and/or the particular location where stent 10 is to be implanted as will be recognized by those skilled in the art upon review of the present disclosure. In view of its possible insertion at a stenotic site within a blood vessel and/or the site of an aneurysm, the exemplary dimensions provided above may be appropriate.

Stent 10 is normally placed in position using a catheter 300 in which stent 10 is inserted into a vessel 100 in a collapsed position. After the tip of the catheter 300 has been positioned within a vessel 100 typically, an artery or vein, stent 10 is released from the catheter 300, positioned, and radially expanded to support the vessel and/or aneurysm. A balloon catheter may be used to expand stent 10 in situ. When a balloon catheter is used to expand the stent, the balloon is generally disposed within the lumen of stent 10 in the contracted condition. Once stent 10 is properly positioned, the balloon is expanded to expand stent 10 into its expanded configuration. In one aspect, the balloon may include a lateral extension to be received and to expand the embolic body 14. Alternatively or in addition to the balloon, stent 10 may be formed from a superelastic material. The superelastic material will cause stent 10 to expand once stent 10 is removed from the lumen of the catheter. That is, the superelastic material configures the stent in an expanded state when the superelastic material is relaxed. Thus, stent body 12 and embolic body 14 can be held in compressed collapsed configuration within the lumen of the catheter by the lumen or by other restraining elements. Once stent body 12 and embolic element 14 are removed from the lumen and/or the other restraining elements release stent body 12 and embolic body 14, stent body 12 and embolic element 14 relax to their expanded state to support the lumen and/or aneurysm. Further, stent body 12 and embolic body 14 may include materials having so-called “shape memory” to radially expand the stent within the lumen. The material known by the trade name “Nitinol” is well known and has superelasticity and shape-memory properties which may facilitate the expansion of stents. The expansion of stent body 12 and embolic body 14 of stent 10 can be accomplished simultaneously or each of stent body 12 and embolic body 14 may be expanded independently of one another as desired by a user.

Once embolic body 14 is positioned within the aneurysm, the embolic body can function to support and/or promote the formation of an embolism within the aneurysm. Embolic body 14 and/or coagulating promoting material 20 within cavity 18 of embolic body 14 may promote coagulation and the formation and securing of an embolism within the aneurysm. In addition, the clotting can be promoted by the process of electro-thrombosis. With electro-thrombosis, either the coagulation promoting material or the embolic body is formed from a conductive material and is electrified. The electrified material promotes the clotting of blood on the conductive material and thereby, forms an embolism. Regardless of whether or not electrothrombosis is used, the embolism typically forms an occlusion sealing off the aneurysm after a relatively brief period.

Stent 10 may be manufactured using various techniques that are known to those skilled in the art with variations for including embolic body 10 evident to those skilled in the art upon review of the present disclosure. Particularly, in one exemplary method, stent 10 may be formed from a continuous tubular blank to be cut up into individual stents, the walls with openings being formed by techniques such as laser cutting, photo-engraving, electron-discharge machining, etc. In another exemplary method, stent 10 may be formed from a strip-like body in which the regions with openings are formed, for example, by the techniques mentioned above, with a view to the subsequent closure of the strip-like element to form a tube. In yet another exemplary method, stent 10 may be formed from metal wire shaped by the successive connection of loops of wire, for example, by means of micro-welding, brazing, gluing, crimping operations, etc.

It should again be pointed out that, although the main application of the stents described relates to the treatment of blood vessels, the use of the present invention as an element for supporting any lumen in a human or animal body can certainly be envisaged. The present invention may be embodied in other specific forms without departing from the spirit or essential attributes thereof, and it is therefore desired that the present embodiment be considered in all respects as illustrative and not restrictive, reference being made to the appended claims rather than to the foregoing description to indicate the scope of the invention.

1. A stent to stabilize an aneurysm, comprising:
a stent body having an outer surface and shaped to be received within a lumen within a patient, wherein the stent body may be configured in at least a collapsed configuration for insertion into the lumen of the patient and an expanded configuration; and
an embolic body secured to the stent body and extending from the outer surface of the stent body to be received within an aneurysm when the stent body is configured in an expanded configuration.

2. A stent, as in claim 1, further comprising the embolic body defining a cavity within the embolic body.

3. A stent, as in claim 2, further comprising a coagulation promoting material secured within the cavity.

4. A stent, as in claim 1, further comprising the stent body having a cylindrical shape;

5. A stent, as in claim 1, further comprising the stent body having a cylindrical shape with a bifurcation at one end of the stent body.

6. A stent system, comprising:
a stent body having an outer surface and shaped to be received within a lumen within a patient, wherein the stent body may be configured in at least a collapsed configuration for insertion into the lumen of the patient and an expanded configuration to contact at least a portion of a vessel wall; and
an embolic body wherein the embolic body configured to be received within an aneurysm to promote thrombosis and secured within the aneurysm by the stent body when the stent body is configured in an expanded configuration.

7. A stent, as in claim 1, further comprising the embolic body defining a cavity within the embolic body.

8. A stent, as in claim 2, further comprising a coagulation promoting material secured within the cavity.

9. A stent, as in claim 1, further comprising the stent body having a cylindrical shape;

10. A stent, as in claim 1, further comprising the stent body having a cylindrical shape with a bifurcation at one end of the stent body.
